(19) United States
(12) Patent Application Publication
(10) Pub. No.: US 2002/0107288 A1
(43) Pub. Date: Aug. 8, 2002

(54) Methods of sterilizing with dipercarboxylic acids

(76) Inventors: Waheguru Pal Singh, Collage Station, TX (US); Anthony Giletto, Collage Station, TX (US); G. Duncan Hitchens, Bryan, TX (US)

Correspondence Address: STREETS & STEELE, P.O. Box 1612, Cypress, TX, 77410-1612, US

(21) Appl. No.: 09/733,611

(22) Filed: Dec. 8, 2000

(51) Int. Cl. A61K 31/19

(52) U.S. Cl. 514/557

(57) ABSTRACT

Dry dipercarboxylic acid material and methods of using dry dipercarboxylic acid particulates to form novel sterilizing solutions or liquid chemical germicides. The dipercarboxylic acids or organic diperoxygen compounds can be synthesized and isolated as solid powders with an extended shelf life. The powders are also soluble in water for quickly preparing liquid disinfectant solutions, whenever and wherever desired, from a potable water source. The dry dipercarboxylic acid materials are selected from diperglutaric acid, diperadipic acid, diperpimelic acid, dipersuberic acid, and diperazelaic acid. Upon dissolution into water, these compounds have demonstrated the ability to inactivate high numbers of spores, including sterilization of medical equipment in 10 minutes at room temperature.

### BACKGROUND OF THE INVENTION

1. Field of the Invention

This invention relates to sterilizing compositions, methods of forming sterilizing solutions, and methods of sterilizing articles using those sterilizing solutions.

2. Background of the Related Art

Peracids are potent biocides that have a broad-spectrum killing potential. They are disinfectants that decompose into chemically benign end products. However, current peracid formulations have many limitations. Formulations usually consist of low molecular weight peracids that have a very pungent odor, which makes their handling difficult and even hazardous. A further considerable disadvantage of low molecular weight peracids are their relatively high volatility, which in addition to resulting in an annoying odor, leads to an undesirable uptake in man, animals, and plants through inhalation and resorption and could preclude their use on toxicological grounds.

Eggensperger et al. in U.S. Pat. No. 4,129,517 identified the odor and volatility of commercial peracid solutions as undesirable and potentially toxic. They identified a need for a stable and odorless concentrated peracid solution. Their invention describes the preparation of a concentrated diperglutaric acid (HOOOC—CH₂—CH₂—CH₂—COOOH) solution to be used as a concentrated liquid from which more dilute disinfecting solutions could be prepared. Their composition contains from 1 to 60 percent diperglutaric acid and from 1 to 50 percent hydrogen peroxide. They also describe that lower molecular weight diperacids (dipermaleic acid, C3 and dipersuccinic acid, C4) did not form stable peracid concentrates and that higher molecular weight diperacids (diperadipic acid, C6) were not soluble enough to prepare an adequate peracid concentrate solution.

Although Eggensperger et al. address the undesirable odor and inhalation hazard of commercially available peracid formulations, their preparation is indicative of another limitation common to commercially available peracid formulations and that is how they are prepared and stored. The classical method used to prepare peracids is oxidation of the corresponding carboxylic acid in the presence of hydrogen peroxide. This reaction is shown in FIG. 1. In the specific case involving the preparation of peracetic acid (the most commonly prepared peracid), glacial acetic acid (R=CH₃) is mixed with concentrated hydrogen peroxide in the presence of sulfuric acid; the products are peracetic acid and water. The reaction does not go to completion but reaches an equilibrium where appreciable amounts of starting material remain in solution.

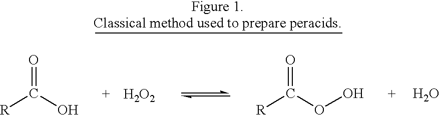

It is not possible to isolate peracetic acid from the reaction mixture because it is unstable. Instead, commercially available preparations of peracetic acid usually consist of an equilibrium mixture of peracetic acid, acetic acid, hydrogen peroxide, and water. This type of liquid formulation has many limitations. Typically, high concentrations of acid and peroxide are required. Commercially available preparations of peracetic acid contain from 7 to 25% hydrogen peroxide and from 6 to 40% acetic acid. Hydrogen peroxide above 6% is a contact hazard, above 15% it can cause severe bums, and higher concentrations can start fires when it comes in contact with combustible material. High concentrations of acetic acid are also hazardous.

Therefore, a need still exists for compositions and methods that provide effective sterilizing solutions without concerns for stability and shelf-life, or transportation of hazardous and bulky solutions. It would be desirable to have a stable, solid peracid formulation that does not contain the reagents or chemicals used in its formation, and that can be dissolved in water or an aqueous solution at the point of sterilization in concentrations high enough to obtain a solution capable of sterilizing articles. It would be further desirable if solutions of the composition had even greater sterilizing power than peracetic acid solutions.

### SUMMARY OF THE INVENTION

The present invention provides stable, solid peracid material that can be dissolved at concentrations high enough to form novel sterilizing solutions or liquid chemical germicides. The dipercarboxylic acids or organic diperoxygen compounds of the present invention can be synthesized and isolated as solid powders with an extended shelf-life. The powders are soluble in water for quickly preparing liquid sterilizing solutions, whenever and wherever desired, from a potable water source. The dry dipercarboxylic acid material is selected from diperglutaric acid (C5), diperadipic (C6), diperpimelic (C7), dipersuberic acid (C8), and diperazelaic (C9). Upon dissolution into water, these compounds have demonstrated the ability to inactivate high numbers of spores in 10 minutes at room temperature. These solutions are characterized by effectiveness against a broad spectrum of microorganisms, including but not limited to mycobacteria, yeasts, fungi, viruses and resistant bacterial spores. These solutions are also effective as cold sterilizing solutions compatible with most instruments and accessories, emit no harmful vapors, have little or no toxicity, and leave only biodegradable byproducts.

To understand the value of the invention, it is imperative to distinguish between sterilization and disinfection. Strictly, sterilization is the use of physical or chemical means to destroy all microbial life. Disinfection is a less lethal process than sterilization in which most, but not all, of the microbial life is destroyed. Therefore, sterilization represents the highest possible level of disinfection. Microbial life is a broad term that encompasses many different organisms including bacterial endospores, mycobacteria, fungi, vegetative bacteria, and viruses. Bacterial endospores are the most resistant to chemical disinfectants and therefore represent the benchmark of microbial organisms in the evaluation of chemical disinfectants. If a chemical disinfectant can reduce the level of bacterial endospores by six logarithms or more it is considered a liquid sterilant. The present invention describes stable, solid peracids that can be dissolved in water at concentrations high enough to be defined as a sterilant.

The stable, solid peracid materials of the present invention is preferably in a form that is rapidly dissolvable. The preferred form of the material is small particles including, but not limted to, powders, colloids, crystals, and tablets. The term “particles” as used herein shall be taken to mean any discrete unit of material structure and should not be limited to a particular particle size or range of sizes.

### BRIEF DESCRIPTION OF THE DRAWINGS

So that the above recited features and advantages of the present invention can be understood in detail, a more particular description of the invention, briefly summarized above, may be had by reference to the embodiments thereof that are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a graph showing Log B. subtilis spore concentration as a function of the exposure time to various saturated dipercarboxylic acid solutions.

FIG. 2 is a graph showing the results of certain zone of inhibition tests.

### DETAILED DESCRIPTION OF THE INVENTION

The present invention relates to a select subgroup of aliphatic dipercarboxylic acids that can be prepared as solids that are stable at room temperature, easier to synthesize, and effective against a variety of pathogenic bacteria and spores. These dipercarboxylic acids are synthesized in a single step reaction, in which hydrogen peroxide solution is added into a solution of the parent dicarboxylic acid dissolved in sulfuric acid. The synthetic scheme is set out in Equation (1).

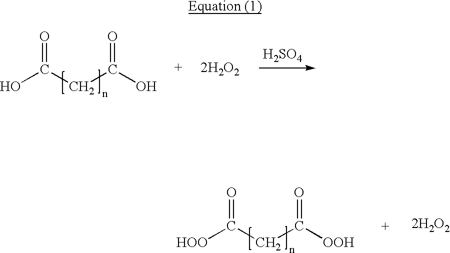

The procedure for preparation of dipercarboxylic acid use starting materials that are common industrial chemicals and are thus commercially available. The process involves addition of hydrogen peroxide into a solution of dicarboxylic acid in sulfuric acid with external cooling, then adding saturated ammonium sulfate to precipitate the dipercarboxilic acids. The precipitates are filtered, dried and are ready to use without any further purification. The yield of dipercarboxylic acids in this reaction is above 85%.

The dipercarboxylic acids of Equation (1) with n=3 to n—5 are fairly soluble in water. They are isolated by diluting the reaction mixture with saturated ammonium sulfate solution at 0° C., followed by filtration. The higher peracids can be precipitated using half-saturated ammonium sulfate. The dipercarboxylic acids have a variable melting decomposition temperature of about 80-100° C. At room temperature, the dipercarboxylic acids are relatively stable.

The sterilizing solutions of dipercarboxylic acids of the present invention are operable at any temperature between the freezing point of the solution and the boiling point of the solution. The activity of the diper acids is believed to be greater at higher temperatures (resulting in faster sterilization), but the decomposition of the diper acids is also greater at higher temperatures. The preferred temperature of the sterilizing solutions is between 0° C. and 50° C., most preferably at a temperature that is ambient, such as 20-30° C., and even more preferably at 25 C.

Peracids are strong oxidizing agents, and have a high affinity for sulfhydryl, sulfide, disulfide, and carbon to carbon double bonds. These bonds play critical roles in the function of certain essential enzymes and of cell membranes. Without limiting the present invention to any particular mechanism, it is believed that oxidative cleavage of these bonds inactivate the enzyme(s) in question and result in the death of the cell. Alternatively, if the affected bonds are part of the cell membrane, then the material transport and osmotic functions of the membrane would be disrupted, again causing death of the cell. Because spore coats are known to have a high concentration of disulfide bonds, disruption of the spore coat by oxidation of disulfide bonds would expose the sensitive interior of the spore to the sterilant and cause spore death.

The entire electron transport system of all living cells is highly susceptible to oxidation, and its disruption would result in rapid cell death. In this context, it is interesting to note that most living cells protect themselves from oxidative damage with enzymes, such as catalase. Catalase very effectively decomposes hydrogen peroxide as soon as it is formed in cells as a result of radiation or some other process. Catalase does not decompose organic peroxides. Organic peroxides deactivate catalase, and can therefore continue their action unhindered, while depriving the cell of an important protective mechanism. Further, peracids can oxidize alcohol, amine, and a variety of other functional groups abound in living cells and are powerful protein denaturants, and that effect will be lethal to all cells, microorganisms, and spores. The relative importance of these various effects will vary from one species to another. While the exact modality by which peracids kill microorganisms, spores, and viruses is not known, any of the mechanisms described above could alone cause death and most if not all probably contribute in causing death.

The select subset of dipercarboxylic acids of the present invention are unique sterilizing agents in that they can be form dry solid particulates, yet they can still be readily dissolved in water with minimal agitation, such as stirring. As dry solid particulates, the dipercarboxylic acids can be stored for extended periods without degradation. It is preferred that the dry solid dipercarboxylic acids be stored in the absence of other organic compounds that could be oxidized by the acids. However, many saturated organic compounds may not be oxidized and may therefore be included in formulations to improve dissolution of the material into water. Examples of suitable saturated organic compounds include long chain aliphatic fatty acids, long chain aliphatic quaternary ammonium salts, or combinations thereof. It is also preferred that the dipercarboxylic acids are dissolved with stirring, but without heating, without using special solubilizers, and without using special solvents. Accordingly, dissolution into water or an aqueous solution produces a very effective sterilizing solution in situ within equipment or in the field under austere environments.

Where necessary, insoluble peracids can be suspended by the use of a combination of a C12-C15 primary alcohol ethoxylate having 7 ethylene oxides, alkylbenzene suphonate and very high levels (>6% w/w) of an electrolyte such as sodium sulphate. Insoluble peracids can also be suspended by a C12-C14 alcohol ethoxylate having 7.5 ethoxylates in combination with sodium dodecylbenzene sulphonate, but the pH of these compositions must be maintained between 3.5 and 4.1. A third solution for suspending insoluble peracids is a C12-C15 alcohol ethoxylate having 3 ethoxylates in combination with a secondary alkane sulphonate and 10% w/w sodium sulphate.

The solubility of diperacids in water can be effected by changing the hydrophobicity of the alkyl chain present in the molecule. Solubility of large chain diperacids like dipersabacic acid in water can be enhanced by incorporation of polar functional groups in the carbon chain. Some examples of such groups are hydroxyl, amino, amido, alkoxy, carbonyl, and the like or combinations thereof. These groups can be attached at any or all postions within the alkyl chain of the less soluble diacids.

The stability of peracids improves by avoiding impurities and also by adding stabilizers, preferably inorganic salts. Examples of suitable stabilizers include, but are not limited to, stannates, dipicolinic acid, pyrophosphoric and polypyrophosphoric acids and their salts. The effectiveness of chemical sterilizers is sometimes reduced due to presence of organic load left on the medical/dental instruments. As a result a pre-washing step is generally recommended to improve the degree of sterilization.

The peracid formulations may optionally include an exothern control agent admixed with the diperacid. The water level present in the diperacid-exotherm control composition is also carefully adjusted so that minimum destabilization of the diperacid is brought about by its presence, yet the exotherm control effects are maintained. The preferred exotherm control agents are Na₂SO₄, MgSO₄ and combinations thereof, each being in the hydrated form. Hydrated alkali metal or alkaline earth metal salts may also be used as a means to control the exothermal deterioration of peracids. The diperacids and the stabilizing agents are preferably prepared as distinct granular components of the total composition.

The efficacy of dipercarboxylic acids as broad-range sterilizing agents is demonstrated in the following Examples in which diperglutaric acid is shown to kill a variety of pathogenic bacteria as well as spores.

### EXAMPLE 1

### Synthesis of Dipercarboxylic Acids

Dipercarboxylic acids were synthesized by dissolving 0.05 moles of dicarboxylic acid in 30 grams of 95% sulfuric acid in an open beaker. With good stirring, 13.5 grams (0.2 mole) of 50% hydrogen peroxide was added dropwise over 10-15 minutes keeping the internal temperature between 0 and 20° C. using an ice bath. Stirring was continued for an additional 3 hours. Adding several volumes of saturated aqueous ammonium sulfate then precipitated the dipercarboxylic acid, such as 10 grams of 85% dipercarboxylic acid.

The precipitate was washed several times until the filtrate was relatively free of sulfuric acid. The crude product was dried overnight in a vacuum oven at room temperature. The dried product was then dissolved in ethanol and recrystallized by gradual addition of water. The recrystallized dipercarboxylic acid was filtered and dried again in the vacuum oven over night at room temperature to obtain the desired solid particulate of dipercarboxylic acid. The recrystalized samples can be used to determine proton NMR, FTIR, mass as well as elemental analysis.

### EXAMPLE 2

### Sterilization Rates of Dipercarboxylic Acids

A crude experiment was done to first estimate the solubility in water of diperglutaric acid (C5), dipersuberic acid (C8), and dipersebacic acid (C10) prepared in accordance with Example 1. It was estimated that the limit of solubility of these peracids in water was 10%, 0.8%, and 0.1% wt/v for diperglutaric, dipersuberic, and dipersebacic, respectively.

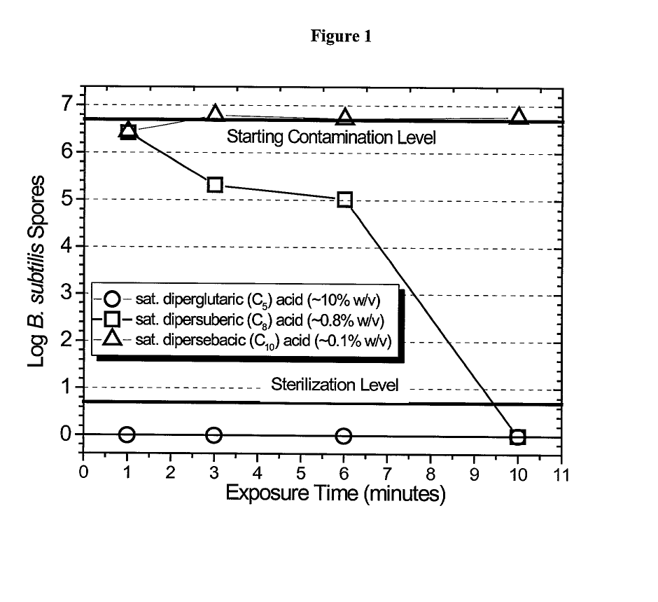

A saturated solution of each peracid was prepared in water. 1.2 mL of saturated peracid solution was placed in a 2 mL eppindorf tube. At t=0, 0.3 mL of a 2.5×10⁸ spores per mL solution was placed in the eppindorf tube and mixed. The final spore concentration was 1.7×10⁸ spores per mL. At various time points, a 0.2 mL aliquot (containing 3.3×10⁷ spores) was removed from from the eppindorf and added to 0.4 mL of a 10% sodium thiosulfate, 10% bovine serum albumin solution. This solution quenches unreacted peracid. The final spore concentration was 5.6×10⁷ spores per mL. Dilutions were made and 0.1 mL (5.6×10⁶ spores) of each was plated on nutrient agar plates. The plates were incubated at 37° C. overnight and colonies were counted the next day to determine the number of spores that survived exposure to peracid. The log of the number of spores plated (5.6×10⁶) is 6.74. In FIG. 1, this value is plotted in the graph as a dark line and referred to as the “Starting Contamination Level”. “Sterilization Level” which is the dark line near the bottom of the graph is simply the “Starting Contamination Level” minus 6. The X-axis in the graph is exposure time of the spores to peracid.

### EXAMPLE 3

### Zones of Inhibition

Zone of inhibition tests are qualitative screens for the inhibitory effect of the compound being tested. Clear zones created by a compound on a bacterial lawn indicates bacteriostatic ability and possible bactericidal capability and the size of the zone of inhibition is a semi-qualitative measure of the strength of the compound. The procedure involved creating lawns of bacteria by spreading 100 μL of broth culture evenly on nutrient agar plates. The bacteria were drawn from broth cultures that had recently reached maximum density. The organisms were Staphylococcus aureus, Psuedomonas aeruginosa, and Escherichia coli. Sterile, 6 mm, white paper discs were placed in the middle of each bacterial lawn. 20 μL of treatment were dispensed onto the surface of each disc. The treatments were: 1.0% and 0.033% diperglutaric acid, 1.0% glutaric acid in water. Each treatment was performed in duplicate for each organism. The plates were incubated at 37° C. for 18-24 hours. All zones were then measured in millimeters across the diameter of the zone of inhibition.

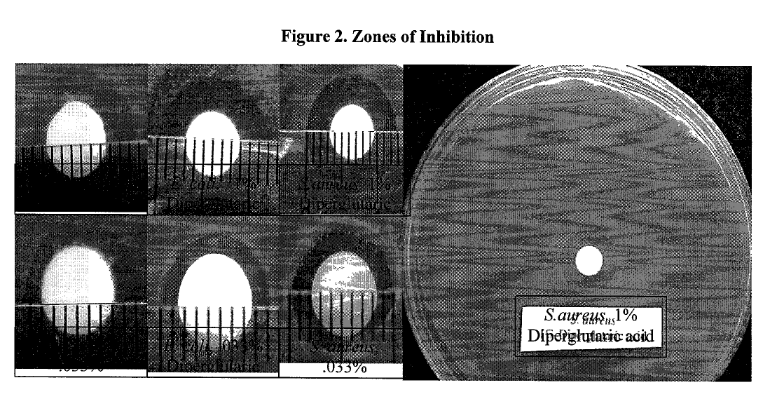

The results of the zone of inhibition study in Table 1 show that diperglutaric acid at 1% in water is very effective in preventing the growth of vegetative cells. Photographs of the zones of inhibition are shown in FIG. 2.

TABLE 1
Zones of Inhibition

| | Diameter of Zones of Inhibition (duplicate experiments) | | |
|---|---|---|---|
| | 1% Glutaric | 1% Diperglutaric | 0.033% Diperglutaric |
| Organism | acid | acid | acid |
| S. aureus | No zone | 13 mm, 13 mm | 10 mm, 9 mm |
| E. coil | No zone | 12 mm, 13 mm | 10 mm, 10 mm |
| P. aeruginosa | No zone | 12 mm, 12 mm | 10 mm, 11 mm |

### EXAMPLE 4

### Biopsy Punch

Biopsy punch enumeration is an extension of the zone of inhibition test, which involves enumerating organisms on the surface or within a removed core (punch). This test provides a quantitative analysis of the viable organisms remaining after treatment. This procedure was carried out exactly as the zone of inhibition testing. After incubation, however, the disc was removed from the plate and a 6-mm core was taken with a sterile, disposable biopsy punch precisely in the location where the disc had been removed. The same three organisms were used and the following treatments were sampled in duplicate: 1.0% diperglutaric acid and 1.0% glutaric acid in water. The core of each plate was aseptically placed in a microcentrifuge tube with 1 ml of sterile 0.85% saline solution and placed on a vortex for 5 minutes. These samples were diluted and plated in duplicate on nutrient agar and allowed to incubate at 37° C. for 18-24 hours for enumeration.

Table 2 shows the results of the biopsy punch experiments, confirming the antimicrobial properties of diperglutaric acid compared to the unreacted parent compound. In conclusion, a 1% diperglutaric acid solution in water has a high potential to be used as a broad spectrum high level disinfectant.

TABLE 2
Biopsy Punch Enumeration

| | | Log₁₀ predisinfection | | Log₁₀ reduction |
|---|---|---|---|---|
| | | count (cfu/6 mm | Log₁₀ postdisinfection count | (cfu/6 mm |
| Treatment | Organism | diameter sample) | (cfu/6 mm diameter sample) | diameter sample) |
| 1% Glutaric | P. aeruginosa | 7.53 | 7.57 | 0.00 |
| acid | S. aureus | 8.11 | 7.48 | 0.63 |
| | E. coli | 7.44 | 6.75 | 0.69 |
| 1% | P. aeruginosa | 7.53 | ND | 7.53 |
| Dipergiutaric | S. aureus | 8.11 | ND | 8.11 |
| acid | E. coli | 7.44 | ND | 7.44 |

### EXAMPLE 5

### Additional Sporicidal Testing

Additional spore testing experiments were done with diperglutaric acid. In this example, the diperglutaric acid was dissolved in a 90% water and 10% ethanol solution and used to kill Bacillus subtilis spores. This experiment was done to demonstrate that an organic solvent can be used in the preparation of sporicidal formulation. Sporicidal capabilities of diperglutaric acid were tested at various concentrations. These procedures called for a 30-minute treatment of Bacillus subtilis spores heat fixed to glass slides. Glass slides were cut in half lengthwise. A suspension of spores, obtained from Steris Corporation of Mentor, Ohio (order # NaO26) in 10% bovine serum albumin (as a simulated organic load) was prepared at a concentration of 1.2×10⁸ spores per mL. 100 μL of this suspension was heat-fixed to each glass slide. These slides were immersed in the following dilutions of the diperglutaric acid: 2%, 1%, 0.3%, 0.1%, and 0.03%.

Control slides were treated in glutaric acid: 2% and 1% in a 10% ethanol/water solution. All treatment concentrations were tested in duplicate. The spore coupons were immersed in 30 ml of test solution for 30 minutes. Following the treatment, the slides were rinsed in sterile water to remove residual acid. Each slide was then placed in a sterile 15-ml test tube containing 2 ml of sterile water. These tubes were sonicated for one hour to resuspend all spores. The sonicated slides were removed from the test tubes. The remaining solutions were serially diluted, plated in duplicate on nutrient agar and incubated for 18-24 hours at 37° C.

TABLE 3
Sporicidal Testing

| | Conc. of | Log₁₀ pre- | Log₁₀ post- | Average log₁₀ | Average log₁₀ |
|---|---|---|---|---|---|
| | active | disinfection count | disinfection count | postdisinfection | reduction |
| Treatment | ingredient | (cfu/coupon) | (cfu/coupon) | count (cfu/coupon) | (cfu/coupon) |
| Glutaric acid in | 2.0% | 6.69 (n = 12) | 6.27, 6.24 | 6.26 | 0.43 |
| 10% EtOH | 1.0% | ″ | 6.26, 6.19 | 6.23 | 0.46 |
| Diperglutaric | 2.0% | ″ | ND,ND | <1.0 | 6.69 |
| acid in 10% | 1.0% | ″ | ND,ND | <1.0 | 6.69 |
| EtOH | 0.33% | ″ | 3.62, 3.52 | 3.57 | 3.12 |
| | 0.1% | ″ | 4.50, 4.94 | 4.72 | 1.97 |
| | 0.033% | ″ | 5.25, 4.73 | 4.99 | 1.70 |
| Glutaraldehyde | 2.0% | 6.06 (n = 3) | 3.95, 2.16, 3.19 | 3.10 | 3.05 |

ND = None Detected

Table 3 shows the results of the spore deactivation study. The control values (pre-disinfection counts) were obtained from spore carriers immersed in sterile distilled water for 30 minutes prior to rinsing, recovery, and enumeration. The average log₁₀ recovery from these controls was log₁₀6.69 per carrier. This compares favorably with the initial number of spores added, which was log₁₀7.08. The number of viable spores recovered was 40% of the number initially applied. Thus losses due to drying, rinsing and sonication do not significantly affect spore viability. Losses of 90% or less are generally considered acceptable in this type of experiment. Glutaric acid, at concentrations of 1% and 2% for 30 minutes, was not an effective sporicidal agent. A log reduction of less than log₁₀0.5 cfu/carrier was achieved. Thus, the unreacted parent carboxylic acid has little effect on spores. In contrast, no viable spores were recovered from carriers that were exposed to diperglutaric acid at 1% and 2% for 30 minutes. Table 3 shows that diperglutaric acid at these concentrations was significantly better at killing spores than that from fleshly prepared 2% glutaraldehyde preparation. Even at a concentration of 0.33%, the diperglutaric acid's effect on spores was similar to that of 2% glutaraldehyde. The results in Table 2 show that diperglutaric acid solutions are highly sporicidal.

In accordance with the above procedures, dipercarboxylic acids can be obtained in greater than 95% purity. Being solids, the dipercarboxylic acids can be dried, freed of gases, and stored under vacuum, as and when desired. Dipercarboxylic acids are also more stable than their counterpart mono-peracids, in particular peracetic acid, and can perform cold sterilization under austere environments, such as where there is a lack of sophisticated equipment.

It is anticipated that the dipercarboxylic acid solutions will be suitable for use in endoscope reprocessors. The contaminated lumens of the scopes are mounted in the reprocessor with or without the usual manual brushing steps. Preferably, the endoscope is subject to multiple cleaning/disinfection cycles in an automated endoscope reprocessor having the disinfection tank of the reprocessor filled with an appropriate dipercarboxylic acid solution. It is believed that dipercarboxylic acid solutions will not damage even the most delicate medical instruments.

The term “comprising” means that the recited elements or steps may be only part of the device and does not exclude additional unrecited elements or steps.

It will be understood that certain combinations and sub-combinations of the invention are of utility and may be employed without reference to other features in sub-combinations. This is contemplated by and is within the scope of the present invention. As many possible embodiments may be made of this invention without departing from the spirit and scope thereof, it is to be understood that all matters hereinabove set forth or shown in the accompanying drawings are to be interpreted as illustrative and not in a limiting sense.

We claim:

1. A method of preparing a sterilizing solution, comprising:
(a) storing dry solid material comprising one or more dipercarboxylic acid; and
(b) dissolving the dry solid material into water as needed to prepare an aqueous sterilizing solution having a dipercarboxylic acid concentration between about 0.1 weight percent and saturation.

2. The method of claim 1, wherein the solid material further comprises inorganic salts.

3. The method of claim 2, wherein the inorganic salts are provided in a stabilizing amount.

4. The method of claim 1, wherein the solid material is substantially free from organic compounds other than the one or more dipercarboxylic acid.

5. The method of claim 1, wherein the one or more dipercarboxylic acid is soluble in water in the absence of a solubilizer.

6. The method of claim 1, wherein the sterilizing solution is substantially free of hydrogen peroxide.

7. The method of claim 1, wherein the one or more dipercarboxylic acid is selected from diperglutaric acid, diperadipic acid, diperpimelic acid, dipersuberic acid, and diperazelaic acid, and combinations thereof.

8. The method of claim 1, wherein the amount of solid material dissolved into water is sufficient to be sporicidal.

9. The method of claim 1, wherein the amount of solid material dissolved into water is sufficient to be sterilizing.

10. The method of claim 1, wherein the water is at ambient temperature.

11. The disinfecting solution formed by the method of claim 1.

12. The method of claim 1, further comprising:
(c) synthesizing one or more dipercarboxylic acid; and
(d) isolating the one or more dipercarboxylic acid form.

13. The method of claim 1, further comprising:
(c) dissolving one or more dicarboxylic acid in sulfuric acid;
(d) reacting the dicarboxylic acid with hydrogen peroxide to form dipercarboxylic acid;
(e) adding ammonium sulfate to precipitate the dipercarboxylic acid;
(f) washing the dipercarboxylic acid to remove sulfuric acid; and
(g) drying the dipercarboxylic acid.

14. The method of claim 13, further comprising:
(h) dissolving the dry dipercarboxylic acid in ethanol; and
(i) recrystallizing the dipercarboxylic acid by gradual addition of water; and
(j) filtering and drying the recrystallized dipercarboxylic acid to obtain solid particles of the dipercarboxylic acid.

15. The method of claim 13, wherein the ratio of hydrogen peroxide to dicarboxylic acid is about 4.

16. The method of claim 13, further comprising
(h) maintaining the reaction temperature between 0 and 20° C.

17. The method of claim 1, wherein the dry solid material further comprises one or more organic solubilizers selected from long chain aliphatic fatty acids, long chain aliphatic quaternary ammonium salts, and combinations thereof.

18. A composition consisting of solid material that comprise one or more dipercarboxylic acid that is solid at room temperature and soluble at sterilizing concentrations in water.

19. The composition of claim 18, wherein the particles form a powder.

20. The composition of claim 18, wherein the one or more dipercarboxylic acid is selected from diperglutaric acid, diperadipic acid, diperpimelic acid, dipersuberic acid, and diperazelaic acid, and combinations thereof.

21. The method of claim 18, wherein the solid particles further comprise inorganic salts.

22. The method of claim 18, wherein the particles further comprise a dipercarboxylic acid-stabilizing amount of inorganic salts.

23. The method of claim 18, wherein the solid particles are substantially free from organic compounds other than the one or more dipercarboxylic acid.

24. The method of claim 18, wherein the one or more dipercarboxylic acid is soluble in water in the absence of a solubilizer.

25. The method of claim 22, wherein the inorganic salts are selected from sodium sulfate, magnesium sulfate, hydrated alkali metal salts, alkaline earth metal salts, and combinations thereof.
